(19) United States
(12) Patent Application Publication
(10) Pub. No.: US 2024/0025937 A1
(43) Pub. Date: Jan. 25, 2024

(54) PREPARATION METHOD OF PEONIDIN-ACYLATED ANTHOCYANIN AND MALVIDIN-ACYLATED ANTHOCYANIN

(71) Applicant: ZHEJIANG UNIVERSITY, Hangzhou (CN)

(72) Inventors: Wei CHEN, Hangzhou (CN); Yang XU, Hangzhou (CN); Jiahong XIE, Hangzhou (CN); Haoxin CUI, Hangzhou (CN)

(21) Appl. No.: 18/026,321

(22) PCT Filed: Jan. 7, 2021
(86) PCT No.: PCT/CN2021/070636

§ 371 Date: Mar. 14, 2023

(30) Foreign Application Priority Data
Sep. 14, 2020 (CN) 202010961038.0

(51) Int. Cl. C07H 17/065; C07H 1/08; B01D 15/16; B01D 15/24; B01D 15/18; B01D 11/04

(52) U.S. Cl. CPC C07H 17/065; C07H 1/08; B01D 15/166; B01D 15/24; B01D 15/1871; B01D 11/0492

(57) ABSTRACT

The present disclosure provides a preparation method of peonidin-3-O-(6-p-coumaryl)glucoside and malvidin-3-O-(6-p-coumaryl)glucoside by separation. In the present disclosure, the preparation method includes alcohol extraction and concentration, macroporous resin purification, extraction, purification by preparative liquid chromatography, and separation by high-speed countercurrent chromatography (HSCCC). In this way, two high-purity monomers, peonidin-acylated anthocyanin and malvidin-acylated anthocyanin, are obtained by separation and purification from grapes. With the preparation method, at least 60 mg of the peonidin-3-O-(6-p-coumaryl)glucoside and at least 200 mg of the malvidin-3-O-(6-p-coumaryl)glucoside can be obtained from 10 kg of grape skins, both of which have a purity of not less than 98%. The preparation method has simple operations, a high processing capacity, and desirable repeatability, and provides a new idea for the development and utilization of grape resources.

### TECHNICAL FIELD

The present disclosure relates to the technical field of separation and purification of natural products, in particular to a preparation method of peonidin-3-O-(6-p-coumaryl)glucoside and malvidin-3-O-(6-p-coumaryl)glucoside by separation.

### BACKGROUND

Anthocyanins are one of the important polyphenols in grapes. Studies have shown that grapes contain anthocyanins derived from the combination of anthocyanidin aglycones with glucose, such as delphinidin, malvidin, peonidin, and petunidin. Recent researches have confirmed that anthocyanins from natural fruits and vegetables have biological activities such as antioxidant, anti-tumor, obesity control, and cardiovascular disease prevention. However, anthocyanins are sensitive to light, temperature, and pH, leading to relatively unstable chemical properties. Moreover, the anthocyanins have low bioavailability. These greatly limit the practical application of anthocyanins. Studies have shown that compared with non-acylated anthocyanins, acylated anthocyanins have biological activities, as well as more stable chemical properties and higher bioavailability. Grapes contain a large amount of p-coumaryl anthocyanins, showing that grape anthocyanins have a better market prospect.

In recent years, new purification techniques have been developed and applied, including solid phase extraction (SPE), preparative high-performance liquid chromatography (preparative-HPLC), and high-speed countercurrent chromatography (HSCCC). HPLC is a chromatographic technique based on the solid-liquid adsorption. In HPLC, adsorbents such as silica gel are used as a stationary phase, and different compound molecules are separated according to the difference in their binding capacities to the stationary phase. The separation effect of HPLC mainly depends on the properties of a stationary phase filler (such as composition and particle size), and whether there is a suitable liquid chromatography method. The HPLC has desirable stability, reliability, and repeatability. Countercurrent chromatography is a liquid-liquid chromatography technique in which both the stationary and mobile phases are liquid. In the countercurrent chromatography, different compound molecules are separated by differences in their partition coefficients between the stationary and mobile phases. The separation effect of countercurrent chromatography mainly depends on the suitability of an adopted two-phase solvent system. Countercurrent chromatography has simple sample pretreatment, wide application range, less sample loss, and high processing capacity.

At present, during the preparation of grape anthocyanins, separation and purification is conducted mainly by extraction, macroporous resin and single column chromatography, or chromatographic techniques. For example, Patent CN104177460A disclosed a method for preparing a 3,5-disaccharide anthocyanin. In this method, ultrasound-assisted extraction, extraction, and purification are conducted. However, an obtained product is an anthocyanin mixture including three different disaccharide anthocyanins, and the product does not involve acylated anthocyanins.

As another example, Patent CN102229633A disclosed a method for separating five kinds of high-purity anthocyanin monomers from grape skins. In this method, five kinds of anthocyanins are obtained by extraction, macroporous resin purification, and preparative liquid chromatography. However, the two-step preparative liquid-phase purification results in sample loss to reduce a purification yield. Moreover, the two acylated anthocyanins (malvidin-acetylated glucoside and malvidin trans-coumaroylated glucoside) have relatively low purities of only 91.7% and 95.5%, respectively.

Patent CN108976268A disclosed a method for preparing two main anthocyanin standard products from Vitis davidii. In this method, adsorption and enrichment of turbid Vitis davidii juice is conducted with macroporous resin. After elution and freeze-drying, a crude anthocyanin product is obtained. By the HSCCC, separation is conducted using a two-phase solvent system including water, n-butanol, methyl tert-butyl ether, acetonitrile, and trifluoroacetic acid (at a volume ratio of 5:4:1:2:0.001 or 5:3:1:1:0.001). In this way, two kinds of anthocyanins are obtained with purities of and 92.2%, respectively. However, from an HPLC chart of the turbid Vitis davidii juice, it can be seen that there is a simple anthocyanin composition. From the limitations of HSCCC, it can be inferred that when an isolated sample has complex anthocyanin components, it may be difficult to obtain the target anthocyanins using this method.

Due to the difficulty in separation and purification of the acylated anthocyanins, there is currently no commercial acylated anthocyanin standard on the market. Accordingly, the research and development of a process for purifying acylated anthocyanin monomers from complex anthocyanin raw materials such as grapes is of great significance for promoting the marketization of anthocyanin standards and the development of deep-processing grape products.

### SUMMARY

Aiming at the deficiencies in the field, the present disclosure provides a preparation method of peonidin-3-O-(6-p-coumaryl)glucoside and malvidin-3-O-(6-p-coumaryl)glucoside by separation. In the present disclosure, according to the characteristics of acylated anthocyanins, the combination of preparative liquid chromatography and HSCCC can achieve the large-scale preparation of high-purity peonidin-3-O-(6-p-coumaryl)glucoside and malvidin-3-O-(6-p-coumaryl)glucoside. The present disclosure provides a new idea for the development and utilization of Chinese grape resources.

The present disclosure provides a preparation method of peonidin-3-O-(6-p-coumaryl)glucoside and malvidin-3-O-(6-p-coumaryl)glucoside by separation, including the following steps:
(1) alcohol extraction and concentration: conducting extraction on grapes serving as a raw material via an acidic alcohol solution, and conducting concentration to obtain a crude extract of grape skin anthocyanin;
(2) macroporous resin purification: injecting the crude extract of grape skin anthocyanin into a macroporous resin, and conducting elution and concentration to obtain an anthocyanin eluate;
(3) extraction: conducting extraction on the anthocyanin eluate using an organic solvent, followed by vacuum concentration, and freeze-drying to obtain a freeze-dried anthocyanin powder;
(4) purification by preparative liquid chromatography: dissolving and injecting the freeze-dried anthocyanin powder into a preparative liquid chromatography system, and conducting detection with an ultraviolet detector under the following specific parameters:
a mobile phase: pure acetonitrile as a phase A, and an aqueous formic acid solution with 1% to 2% of formic acid by volume concentration as a phase B;
a gradient elution program: 0 min to 4 min, 5% to 20%, phase A; 4 min to 18 min, 20% to 25%, phase A; 18 min to 21 min, 25% to 35%, phase A; 21 min to 24 min, 35% to 60%, phase A; 24 min to 27 min, 60% to 5%, phase A; and 27 min to 30 min, 5%, phase A; and
a flow rate of 8 mL/min to 10 mL/min, a column temperature of 30° C., and a detection wavelength of 520 nm; and
collecting a fraction with a retention time of 22.0 min to 23.5 min according to a liquid chromatogram, and conducting vacuum evaporation and freeze-drying to obtain a crude anthocyanin monomer; and
(5) separation by high-speed countercurrent chromatography (HSCCC): mixing ethyl acetate, water, and trifluoroacetic acid at a volume ratio of 1:1:0.001 to obtain a mixture serving as a two-phase solvent system, and using an upper phase as a stationary phase and a lower phase as a mobile phase; pumping the stationary phase and the mobile phase into a HSCCC instrument sequentially; after the stationary phase and the mobile phase reach an equilibrium in a pipeline, dissolving the crude anthocyanin monomer in the mobile phase, conducting sample injection, and conducting detection under the ultraviolet detector at a detection wavelength of 280 nm; collecting fractions with retention times of 116 min to 126 min and 90 min to 100 min, respectively, and conducting vacuum concentration and freeze-drying to obtain the peonidin-3-O-(6-p-coumaryl)glucoside and the malvidin-3-O-(6-p-coumaryl)glucoside, respectively.

In the present disclosure, unless otherwise specified, the percentages of raw materials refer to volume percentage concentrations; unless otherwise specified, all solutions use water as a solvent.

Further, in step (1), a process of the alcohol extraction and concentration specifically includes: washing the grapes and collecting grape skins, mixing the grape skins with an acidic ethanol solution evenly, and conducting pulping, (Prominent) ultrasonic extraction at not more than 50° C. (preferably at a room temperature), and filtration; and subjecting a resulting filtrate to vacuum concentration at to 50° C. to remove ethanol, to obtain the crude extract of grape skin anthocyanin; where
the grape skins and the acidic ethanol solution are at a solid-liquid ratio of 1 g:(4-8) mL;
the acidic ethanol solution has 50% to 80%, preferably 60% to 70% of the ethanol and 0.1% to 1% of an acid by volume concentration; and
the ultrasonic extraction is conducted for 40 min to 120 min.

Further, in step (1), in the acidic ethanol solution, the acid is at least one selected from the group consisting of hydrochloric acid, formic acid, acetic acid, and oxalic acid.

Further, in step (2), a process of the macroporous resin purification specifically includes:
injecting the crude extract of grape skin anthocyanin into the macroporous resin; conducting elution with four times a column volume (4BV) of the acidic ethanol solutions at ethanol volume concentrations of 0, 5%, 20%, 40%, and 60% sequentially; collecting acidic ethanol eluates with the ethanol volume concentrations of 40% and 60%, respectively; and conducting vacuum evaporation at to 50° C. to remove ethanol to obtain the anthocyanin eluate; where
the macroporous resin is preferably selected from the group consisting of AB-8, D101, XAD-7, HPD-100, and DM-130, and has a specific surface area of 450 m²/g to 550 m²/g, an average pore size of 10 nm to 50 nm, and a particle size of 0.3 mm to 1.25 mm; and
in step (2), the acidic ethanol solution is an ethanol solution with 0.1% to 1.5% of an acid by volume concentration, and the acid is at least one selected from the group consisting of hydrochloric acid, formic acid, acetic acid, and oxalic acid.

Further, in step (3), the organic solvent is ethyl acetate.

Further, in step (3), the extraction is preferably conducted more than two times with the organic solvent and the anthocyanin eluate at a volume ratio of 1:1.

Further, in step (4), the freeze-dried anthocyanin powder can be dissolved in a phase B or water.

Further, in step (4), a liquid chromatography column used in the preparative liquid chromatography system is a C18 column, a single sample injection volume is 10 mg to 40 mg based on the freeze-dried anthocyanin powder, and a volume after the vacuum evaporation is 40% to 70% of a volume before the vacuum evaporation.

Further, in step (5), the HSCCC instrument is stabilized at 20° C. to 30° C., and is connected in a forward manner to conduct forward rotation; the stationary phase is pumped into the HSCCC instrument, a rotational speed of the HSCCC instrument is adjusted to 800 r/min to 950 r/min, the mobile phase is introduced at a flow rate of 2 mL/min, and the stationary phase and the mobile phase are made to reach the equilibrium; and each sample injection volume is 20 mg to 50 mg based on the crude anthocyanin monomer.

Compared with the prior art, the present disclosure has the following advantages:
1. In the present disclosure, a method is provided for the first time for simultaneous separation of the peonidin-3-O-(6-p-coumaryl)glucoside (with a molecular structure shown in FIG. 1) and the malvidin-3-O-(6-p-coumaryl)glucoside (with a molecular structure shown in FIG. 2). The peonidin-3-O-(6-p-coumaryl)glucoside monomer has a yield of not less than 6 mg/kg of the grape skins, and the malvidin-3-O-(6-p-coumaryl)glucoside monomer has a yield of not less than 20 mg/kg of the grape skins. Both the two products have a purity of not less than 98%.
2. In the present disclosure, by combining preparative liquid chromatography with HSCCC, the peonidin-3-O-(6-p-coumaryl)glucoside and the malvidin-3-O-(6-p-coumaryl)glucoside can be prepared in large quantities from grape raw materials with complex polyphenol components. The preparation method has high processing capacity and desirable repeatability, and is convenient for industrialized production.

### BRIEF DESCRIPTION OF THE DRAWINGS

FIG. 1 shows a molecular structure diagram of peonidin-3-O-(6-p-coumaryl)glucoside;

FIG. 2 shows a molecular structure diagram of malvidin-3-O-(6-p-coumaryl)glucoside;

FIG. 3 shows an HPLC chart of a crude anthocyanin extract of grape skins in Example 1;

FIG. 4 shows an HPLC chart of an anthocyanin eluate containing the peonidin-3-O-(6-p-coumaryl)glucoside and the malvidin-3-O-(6-p-coumaryl)glucoside after separation and purification by macroporous resin in Example 1;

FIG. 5 shows an HSCCC chart in Example 1;

FIG. 6 shows an HPLC chart of a final product peonidin-3-O-(6-p-coumaryl)glucoside monomer in Example 1;

FIG. 7 shows an HPLC chart of a final product malvidin-3-O-(6-p-coumaryl)glucoside monomer in Example 1;

FIG. 8 shows a secondary mass spectrum of the peonidin-3-O-(6-p-coumaryl)glucoside and the malvidin-3-O-(6-p-coumaryl)glucoside; and

FIG. 9 shows an HPLC chart of a final product in Comparative Example 2.

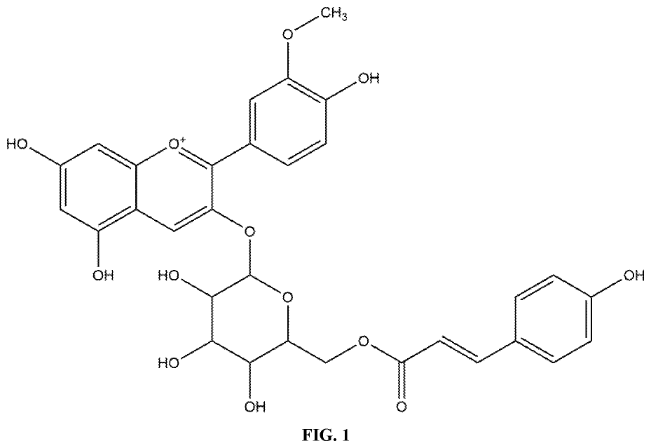

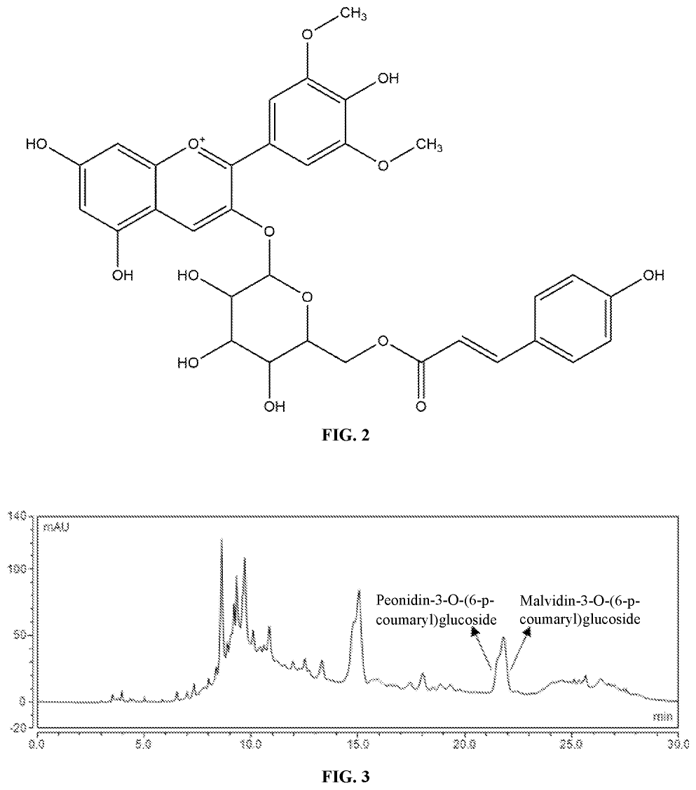

### DETAILED DESCRIPTION OF THE EMBODIMENTS

The present disclosure will be further described below with reference to specific examples. It should be understood that these examples are only intended to illustrate the present disclosure and not to limit the scope of the present disclosure. In the following examples, the experimental methods in which specific conditions are not stated are generally carried out according to conventional conditions or according to the conditions recommended by the manufacturer.

### Example 1

Grapes were washed and peeled to obtain 1 kg of grape skins. The grape skins were thoroughly mixed with a 70% ethanol solution containing 0.5% (v/v) hydrochloric acid at a solid-to-liquid ratio of 1 g:5 mL, ultrasonically extracted for 60 min (at not more than 50° C. in the dark), and then filtered by gauze. An obtained filtrate was centrifuged at 4,000 rpm for 10 min, and a supernatant was collected. The remaining filter residue was extracted once more in a same way to obtain another filtrate. The two filtrates were combined and filtered using a Buchner funnel. An obtained new filtrate was subjected to vacuum evaporation at 45° C. to remove ethanol, and then concentrated to obtain a crude extract of grape skin anthocyanin. The crude extract of grape skin anthocyanin had an HPLC chart shown in FIG. 3.

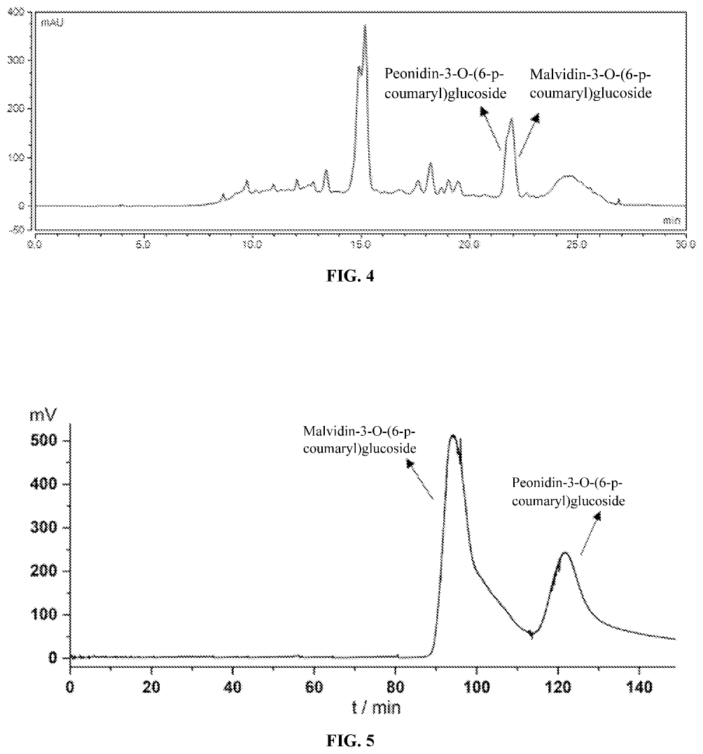

An AB-8 macroporous resin was loaded into a chromatographic column, and washed successively with ethanol, a 0.5 mol/L hydrochloric acid solution, a 0.5 mol/L sodium hydroxide solution, and water. The crude extract of grape skin anthocyanin was injected into the chromatographic column at a flow rate of 0.2 BV/h. After sample injection, elution was conducted with four times a column volume of acidic water (containing 0.5% hydrochloric acid), as well as 5%, 20%, 40%, 60% acidic ethanol (containing 0.5% hydrochloric acid). 40% and 60% acidic ethanol eluates were collected and subjected to vacuum evaporation to remove ethanol. A resulting product was extracted two times with ethyl acetate at a ratio of 1:1. An aqueous phase was appropriately subjected to vacuum concentration, and then freeze-dried to obtain a freeze-dried anthocyanin powder. FIG. 4 showed an HPLC chart of the anthocyanin eluate containing peonidin-3-O-(6-p-coumaryl)glucoside and malvidin-3-O-(6-p-coumaryl)glucoside after separation and purification by macroporous resin.

The liquid-phase separation was conducted by an Ultimate XB-C18 (7 μm, 21.2×250 mm) preparative chromatographic column. A mobile phase included pure acetonitrile (as a phase A) and a 1.5% aqueous formic acid solution (as a phase B). A gradient elution method included: 0 min to 4 min, 5% to 20%, phase A; 4 min to 18 min, 20% to 25%, phase A; 18 min to 21 min, 25% to 35%, phase A; 21 min to 24 min, 35% to 60%, phase A; 24 min to 27 min, 60% to 5%, phase A; and 27 min to 30 min, 5%, phase A, with a flow rate of 10 mL/min, a column temperature of 30° C., and a detection wavelength of 520 nm. The freeze-dried anthocyanin powder was dissolved in the phase B, and subjected to sample injection at a sample injection volume of 4 mL. The fractions collected at 22.0 min to 23.5 min were properly subjected to vacuum concentration, and then freeze-dried to obtain crude products of a peonidin-3-O-(6-p-coumaryl)glucoside monomer and a malvidin-3-O-(6-p-coumaryl)glucoside monomer.

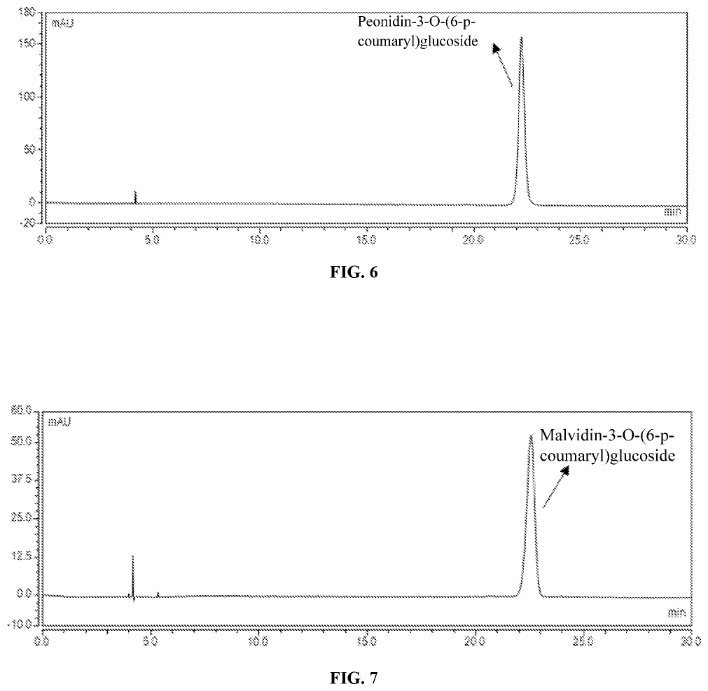

Ethyl acetate, water, and trifluoroacetic acid were placed in a separatory funnel at a volume ratio of 1:1:0.001, shaken well, and allowed to stand for 30 min. The obtained upper and lower phases were separated and degassed by ultrasonic for 30 min separately. The instrument of an HSCCC system was stabilized at 20° C. The stationary phase was pumped into the instrument, and then a rotational speed of the HSCCC instrument was adjusted to 850 r/min. The HSCC instrument was connected in a forward manner to conduct forward rotation, and the mobile phase was fed into the instrument at a flow rate of 2 mL/min until the two phases were equilibrated. The freeze-dried powder of the crude anthocyanin monomer was dissolved in a ratio of the freeze-dried powder to the mobile phase at 3 mg: 1 mL. A resulting solution was filtered with a microporous membrane and then subjected to sample injection, at a single injection volume of 10 mL. The sample was detected under a UV detector at a detection wavelength of 280 nm. Fractions of 90 min to 100 min and 116 min to 126 min were collected, respectively (FIG. 5), subjected to vacuum concentration, and then freeze-dried. In this way, 6.8 mg of the peonidin-3-O-(6-p-coumaryl)glucoside was obtained, with an HPLC chart shown in FIG. 6 and an HPLC purity of 99.3%; and 24 mg of the malvidin-3-O-(6-p-coumaryl)glucoside was obtained, with an HPLC chart shown in FIG. 7 and an HPLC purity of 98.7%.

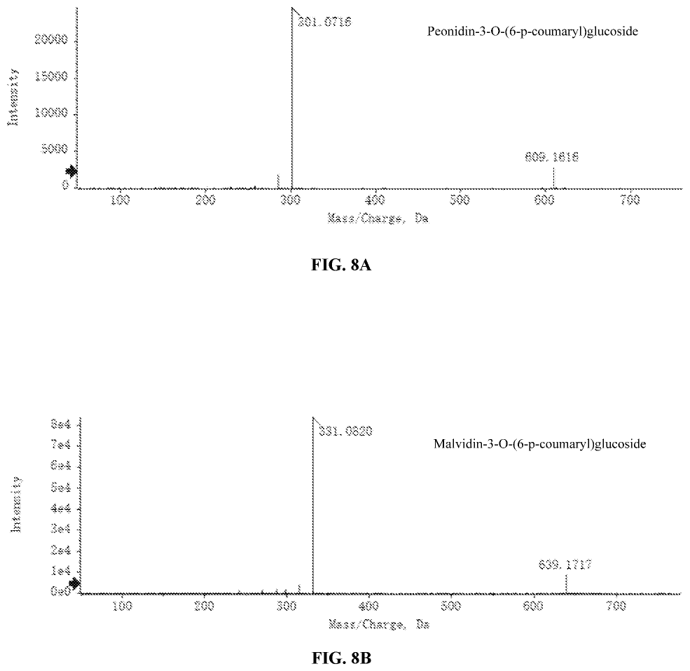

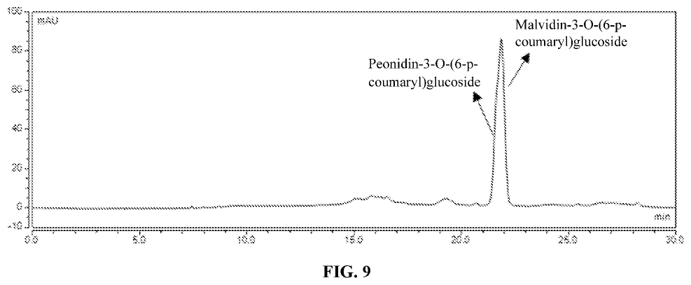

The anthocyanin sample was injected into a mass spectrometer, and then analyzed according to a mass spectrogram (FIG. 8). It was confirmed that the mass number of the separated anthocyanin was normal.

### Example 2

Grapes were washed and peeled to obtain 2 kg of grape skins. The grape skins were thoroughly mixed with a 80% ethanol solution containing 0.5% (v/v) hydrochloric acid at a solid-to-liquid ratio of 1 g:6 mL, ultrasonically extracted for 60 min (at not more than 50° C. in the dark), and then filtered by gauze. An obtained filtrate was centrifuged at 4,000 rpm for 10 min, and a supernatant was collected. The remaining filter residue was extracted once more in a same way to obtain another filtrate. The two filtrates were combined and filtered using a Buchner funnel. An obtained new filtrate was subjected to vacuum evaporation at 45° C. to remove ethanol, and then concentrated to obtain a crude extract of grape skin anthocyanin.

An AB-8 macroporous resin was loaded into a chromatographic column, and washed successively with ethanol, a 0.5 mol/L hydrochloric acid solution, a 0.5 mol/L sodium hydroxide solution, and water. The crude extract of grape skin anthocyanin was injected into the chromatographic column at a flow rate of 0.2 BV/h. After sample injection, elution was conducted with four times a column volume of acidic water (containing 0.5% hydrochloric acid), as well as 5%, 20%, 40%, 60% acidic ethanol (containing 0.5% hydrochloric acid). 40% and 60% acidic ethanol eluates were collected and subjected to vacuum evaporation to remove ethanol. A resulting product was extracted three times with ethyl acetate at a ratio of 1:1. An aqueous phase was appropriately subjected to vacuum concentration, and then freeze-dried to obtain a freeze-dried anthocyanin powder.

The liquid-phase separation was conducted by an Ultimate XB-C18 (7 μm, 21.2×250 mm) preparative chromatographic column. A mobile phase included pure acetonitrile (as a phase A) and a 1.5% aqueous formic acid solution (as a phase B). A gradient elution method included: 0 min to 4 min, 5% to 20%, phase A; 4 min to 18 min, 20% to 25%, phase A; 18 min to 21 min, 25% to 35%, phase A; 21 min to 24 min, 35% to 60%, phase A; 24 min to 27 min, 60% to 5%, phase A; and 27 min to 30 min, 5%, phase A, with a flow rate of 10 mL/min, a column temperature of 30° C., and a detection wavelength of 520 nm. The freeze-dried anthocyanin powder was dissolved in the phase B, and subjected to sample injection at a sample injection volume of 4 mL. The fractions collected at 22.0 min to 23.5 min were properly subjected to vacuum concentration, and then freeze-dried to obtain crude products of a peonidin-3-O-(6-p-coumaryl)glucoside monomer and a malvidin-3-O-(6-p-coumaryl)glucoside monomer.

Ethyl acetate, water, and trifluoroacetic acid were placed in a separatory funnel at a volume ratio of 1:1:0.001, shaken well, and allowed to stand for 30 min. The obtained upper and lower phases were separated and degassed by ultrasonic for 30 min separately. The instrument of an HSCCC system was stabilized at 20° C. The stationary phase was pumped into the instrument, and then a rotational speed of the HSCCC instrument was adjusted to 850 r/min. The HSCC instrument was connected in a forward manner to conduct forward rotation, and the mobile phase was fed into the instrument at a flow rate of 2 mL/min until the two phases were equilibrated. The freeze-dried powder of the crude anthocyanin monomer was dissolved in a ratio of the freeze-dried powder to the mobile phase at 4 mg: 1 mL. A resulting solution was filtered with a microporous membrane and then subjected to sample injection, at a single injection volume of 10 mL. The sample was detected under a UV detector at a detection wavelength of 280 nm. Fractions of 90 min to 100 min and 116 min to 126 min were collected, respectively, subjected to vacuum concentration, and then freeze-dried. In this way, 12 mg of the peonidin-3-O-(6-p-coumaryl)glucoside was obtained, with an HPLC purity of 99.3%; and 43 mg of the malvidin-3-O-(6-p-coumaryl)glucoside was obtained, with an HPLC purity of 98.4%.

### Example 3

Grapes were washed and peeled to obtain 10 kg of grape skins. The grape skins were thoroughly mixed with a 70% ethanol solution containing 0.5% (v/v) hydrochloric acid at a solid-to-liquid ratio of 1 g:4 mL, ultrasonically extracted for 120 min (at not more than 50° C. in the dark), and then filtered by gauze. An obtained filtrate was centrifuged at 4,000 rpm for 10 min, and a supernatant was collected. The remaining filter residue was extracted once more in a same way to obtain another filtrate. The two filtrates were combined and filtered using a Buchner funnel. An obtained new filtrate was subjected to vacuum evaporation at 50° C. to remove ethanol, and then concentrated to obtain a crude extract of grape skin anthocyanin.

An AB-8 macroporous resin was loaded into a chromatographic column, and washed successively with ethanol, a 0.5 mol/L hydrochloric acid solution, a 0.5 mol/L sodium hydroxide solution, and water. The crude extract of grape skin anthocyanin was injected into the chromatographic column at a flow rate of 0.2 BV/h. After sample injection, elution was conducted with four times a column volume of acidic water (containing 0.5% hydrochloric acid), as well as 5%, 20%, 40%, 60% acidic ethanol (containing 0.5% hydrochloric acid). 40% and 60% acidic ethanol eluates were collected and subjected to vacuum evaporation to remove ethanol. A resulting product was extracted two times with ethyl acetate at a ratio of 1:1. An aqueous phase was appropriately subjected to vacuum concentration, and then freeze-dried to obtain a freeze-dried anthocyanin powder.

The liquid-phase separation was conducted by an Ultimate XB-C18 (7 μm, 21.2×250 mm) preparative chromatographic column. A mobile phase included pure acetonitrile (as a phase A) and a 1.5% aqueous formic acid solution (as a phase B). A gradient elution method included: 0 min to 4 min, 5% to 20%, phase A; 4 min to 18 min, 20% to 25%, phase A; 18 min to 21 min, 25% to 35%, phase A; 21 min to 24 min, 35% to 60%, phase A; 24 min to 27 min, 60% to 5%, phase A; and 27 min to 30 min, 5%, phase A, with a flow rate of 10 mL/min, a column temperature of 30° C., and a detection wavelength of 520 nm. The freeze-dried anthocyanin powder was dissolved in the phase B, and subjected to sample injection at a sample injection volume of 4 mL. The fractions collected at 22.0 min to 23.5 min were properly subjected to vacuum concentration, and then freeze-dried to obtain crude products of a peonidin-3-O-(6-p-coumaryl)glucoside monomer and a malvidin-3-O-(6-p-coumaryl)glucoside monomer.

Ethyl acetate, water, and trifluoroacetic acid were placed in a separatory funnel at a volume ratio of 1:1:0.001, shaken well, and allowed to stand for 30 min. The obtained upper and lower phases were separated and degassed by ultrasonic for 30 min separately. The instrument of an HSCCC system was stabilized at 20° C. The stationary phase was pumped into the instrument, and then a rotational speed of the HSCCC instrument was adjusted to 850 r/min. The HSCC instrument was connected in a forward manner to conduct forward rotation, and the mobile phase was fed into the instrument at a flow rate of 2 mL/min until the two phases were equilibrated. The freeze-dried powder of the crude anthocyanin monomer was dissolved in a ratio of the freeze-dried powder to the mobile phase at 5 mg: 1 mL. A resulting solution was filtered with a microporous membrane and then subjected to sample injection, at a single injection volume of 10 mL. The sample was detected under a UV detector at a detection wavelength of 280 nm. Fractions of 90 min to 100 min and 116 min to 126 min were collected, respectively, subjected to vacuum concentration, and then freeze-dried. In this way, 62 mg of the peonidin-3-O-(6-p-coumaryl)glucoside was obtained, with an HPLC purity of 98.5%; and 210 mg of the malvidin-3-O-(6-p-coumaryl)glucoside was obtained, with an HPLC purity of 98.2%.

### Comparative Example 1

In this comparative example, a preparation process was the same as that of Example 1, except that: the extraction was conducted with an acid-free ethanol solution instead of the acidic ethanol solution. Other steps remain unchanged. The target products peonidin-3-O-(6-p-coumaryl)glucoside monomer had a yield of 2 mg/kg of the grape skins, which was much lower than that of 6 mg/kg of the grape skins; malvidin-3-O-(6-p-coumaryl)glucoside monomer had a yield of 7 mg/kg of the grape skins, which was much lower than that of 20 mg/kg of the grape skins.

### Comparative Example 2

In this comparative example, a preparation process was the same as that of Example 1, except that: the HSCCC purification was omitted. Other steps remain unchanged. A resulting final product was a mixture of the peonidin-3-O-(6-p-coumaryl)glucoside and the malvidin-3-O-(6-p-coumaryl)glucoside, and the mixture also included other impurities (FIG. 9).

### Comparative Example 3

In this comparative example, a preparation process was the same as that of Example 1, except that: the solvent system for HSCCC separation was replaced with a system including water, n-butanol, methyl tert-butyl ether, acetonitrile, and trifluoroacetic acid at a volume ratio of 5:4:1:2:0.001. After testing, the target compounds peonidin-3-O-(6-p-coumaryl)glucoside and malvidin-3-O-(6-p-coumaryl)glucoside were mainly retained in the upper phase and could not be obtained.

### Comparative Example 4

In this comparative example, a preparation process was the same as that of Example 1, except that: the solvent system for HSCCC separation was replaced with a system including ethyl acetate, water, and trifluoroacetic acid at a volume ratio of 1:2:0.001. After testing, the target compounds peonidin-3-O-(6-p-coumaryl)glucoside and malvidin-3-O-(6-p-coumaryl)glucoside could not be obtained.

### Comparative Example 5

In this comparative example, a preparation process was the same as that of Example 1, except that: the solvent system for HSCCC separation was replaced with a system including ethyl acetate, water, and trifluoroacetic acid at a volume ratio of 2:1:0.001. After testing, the target compounds peonidin-3-O-(6-p-coumaryl)glucoside and malvidin-3-O-(6-p-coumaryl)glucoside could not be obtained.

In addition, it should be understood that various changes or modifications may be made to the present disclosure by those skilled in the art after reading the above teaching content of the present disclosure, and these equivalent forms also fall within the scope defined by the appended claims of the present disclosure.

What is claimed is:

1. A preparation method of peonidin-3-O-(6-p-coumaryl)glucoside and malvidin-3-O-(6-p-coumaryl)glucoside by separation, comprising the following steps:
(1) alcohol extraction and concentration: conducting extraction on grapes serving as a raw material via an acidic alcohol solution, and conducting concentration to obtain a crude extract of grape skin anthocyanin;
(2) macroporous resin purification: injecting the crude extract of grape skin anthocyanin into a macroporous resin, and conducting elution and concentration to obtain an anthocyanin eluate;
(3) extraction: conducting extraction on the anthocyanin eluate using an organic solvent, followed by vacuum concentration, and freeze-drying to obtain a freeze-dried anthocyanin powder;
(4) purification by preparative liquid chromatography: dissolving and injecting the freeze-dried anthocyanin powder into a preparative liquid chromatography system, and conducting detection with an ultraviolet detector under the following specific parameters:
a mobile phase: pure acetonitrile as a phase A, and an aqueous formic acid solution with 1% to 2% of formic acid by volume concentration as a phase B;
a gradient elution program: 0 min to 4 min, 5% to 20%, phase A; 4 min to 18 min, 20% to 25%, phase A; 18 min to 21 min, 25% to 35%, phase A; 21 min to 24 min, 35% to 60%, phase A; 24 min to 27 min, 60% to 5%, phase A; and 27 min to 30 min, 5%, phase A; and
a flow rate of 8 mL/min to 10 mL/min, a column temperature of 30° C., and a detection wavelength of 520 nm; and
collecting a fraction with a retention time of 22.0 min to 23.5 min according to a liquid chromatogram, and conducting vacuum evaporation and freeze-drying to obtain a crude anthocyanin monomer; and
(5) separation by high-speed countercurrent chromatography (HSCCC): mixing ethyl acetate, water, and trifluoroacetic acid at a volume ratio of 1:1:0.001 to obtain a mixture serving as a two-phase solvent system, and using an upper phase as a stationary phase and a lower phase as a mobile phase; pumping the stationary phase and the mobile phase into a HSCCC instrument sequentially;
after the stationary phase and the mobile phase reach an equilibrium in a pipeline, dissolving the crude anthocyanin monomer in the mobile phase, conducting sample injection, and conducting detection under the ultraviolet detector at a detection wavelength of 280 nm; collecting fractions with retention times of 116 min to 126 min and 90 min to 100 min, respectively, and conducting vacuum concentration and freeze-drying to obtain the peonidin-3-O-(6-p-coumaryl)glucoside and the malvidin-3-O-(6-p-coumaryl)glucoside, respectively.

2. The method according to claim 1, wherein in step (1), a process of the alcohol extraction and concentration specifically comprises: washing the grapes and collecting grape skins, mixing the grape skins with an acidic ethanol solution evenly, and conducting pulping, ultrasonic extraction at not more than 50° C., and filtration; and subjecting a resulting filtrate to vacuum concentration at 40° C. to 50° C. to remove ethanol, to obtain the crude extract of grape skin anthocyanin; wherein
the grape skins and the acidic ethanol solution are at a solid-liquid ratio of 1 g:(4-8) mL;
the acidic ethanol solution has 50% to 80% of the ethanol and 0.1% to 1% of an acid by volume concentration; and
the ultrasonic extraction is conducted for 40 min to 120 min.

3. The method according to claim 2, wherein in the acidic ethanol solution, the acid is at least one selected from the group consisting of hydrochloric acid, formic acid, acetic acid, and oxalic acid.

4. The method according to claim 1, wherein in step (2), a process of the macroporous resin purification specifically comprises:
injecting the crude extract of grape skin anthocyanin into the macroporous resin; conducting elution with four times a column volume of the acidic ethanol solutions at ethanol volume concentrations of 0, 5%, 20%, 40%, and 60% sequentially; collecting acidic ethanol eluates with the ethanol volume concentrations of 40% and 60%, respectively; and conducting vacuum evaporation at to 50° C. to remove ethanol to obtain the anthocyanin eluate; wherein
the macroporous resin is selected from the group consisting of AB-8, D101, XAD-7, HPD-100, and DM-130, and has a specific surface area of 450 m²/g to 550 m²/g, an average pore size of 10 nm to 50 nm, and a particle size of 0.3 mm to 1.25 mm; and
the acidic ethanol solution is an ethanol solution with 0.1% to 1.5% of an acid by volume concentration, and the acid is at least one selected from the group consisting of hydrochloric acid, formic acid, acetic acid, and oxalic acid.

5. The method according to claim 1, wherein in step (3), the organic solvent is ethyl acetate.

6. The method according to claim 1, wherein in step (4), a liquid chromatography column used in the preparative liquid chromatography system is a C18 column, a single sample injection volume is 10 mg to 40 mg based on the freeze-dried anthocyanin powder, and a volume after the vacuum evaporation is 40% to 70% of a volume before the vacuum evaporation.

7. The method according to claim 1, wherein in step (5), the HSCCC instrument is stabilized at to 30° C., and is connected in a forward manner to conduct forward rotation; the stationary phase is pumped into the HSCCC instrument, a rotational speed of the HSCCC instrument is adjusted to 800 r/min to 950 r/min, the mobile phase is introduced at a flow rate of 2 mL/min, and the stationary phase and the mobile phase are made to reach the equilibrium; and each sample injection volume is 20 mg to 50 mg based on the crude anthocyanin monomer.
